(19) United States
(12) Plant Patent Application Publication
(10) Pub. No.: US 2014/0245513 P1
(43) Pub. Date: Aug. 28, 2014

(54) Fuchsia plant named 'FWUWW22-0'

(50) ;

(71) Applicant: Hubertus Volmary, Munster (DE)

(72) Inventor: Hubertus Volmary, Munster (DE)

(21) Appl. No.: 13/815,431

(22) Filed: Feb. 28, 2013

(51) Int. Cl. A01H 5/00

(52) U.S. Cl. PLT/300

(57) ABSTRACT

A new and distinct Fuchsia cultivar named ‘FWUWW22-0’ is disclosed, characterized a compact, upright plant habit, with a large number of flowers, with at least 2 flowers at every axil. Flowers have a very light pink calyx and white corolla, face outward, and flowering response is not dependent upon day length. The new variety is a Fuchsia, typically produced as a garden or container plant.

### LATIN NAME OF THE GENUS AND SPECIES

Fuchsia hybrid

### VARIETY DENOMINATION

‘FWUWW22-0’

### BACKGROUND OF THE INVENTION

The new cultivar is a product of a planned breeding program. The new variety originated from a cross pollination of the seed parent, an unnamed, unpatented proprietary selection of Fuchsia hybrid with the pollen parent a different unnamed, unpatented Fuchsia hybrid. The crossing was made during the Summer of 2009, at a research greenhouse in Munster, Germany. ‘FWUWW22-0’ was discovered by the inventor, Hubertus Volmary, a citizen of Germany, at the same research greenhouse during the Spring of 2010.

Asexual reproduction of the new cultivar ‘FWUWW22-0’ was first performed in Munster Germany, at a commercial greenhouse by vegetative cuttings in Summer of 2010. ‘FWUWW22-0’ has since produced several generations and has shown that the unique features of this cultivar are stable and reproduced true to type.

### SUMMARY OF THE INVENTION

The cultivar ‘FWUWW22-0’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘FWUWW22-0.’ These characteristics in combination distinguish ‘FWUWW22-0’ as a new and distinct Fuchsia cultivar:

1. Compact and upright plant habit.

2. Highly floriferous plants.

3. Flowering response not dependent upon day length.

4. Distinctive pastel pink calyx with white corolla.

5. Double flowers.

6. Flowers more outwardly facing than pendulous.

7. 2 or more flowers emerging from each axil.

### BRIEF DESCRIPTION OF THE PHOTOGRAPHS

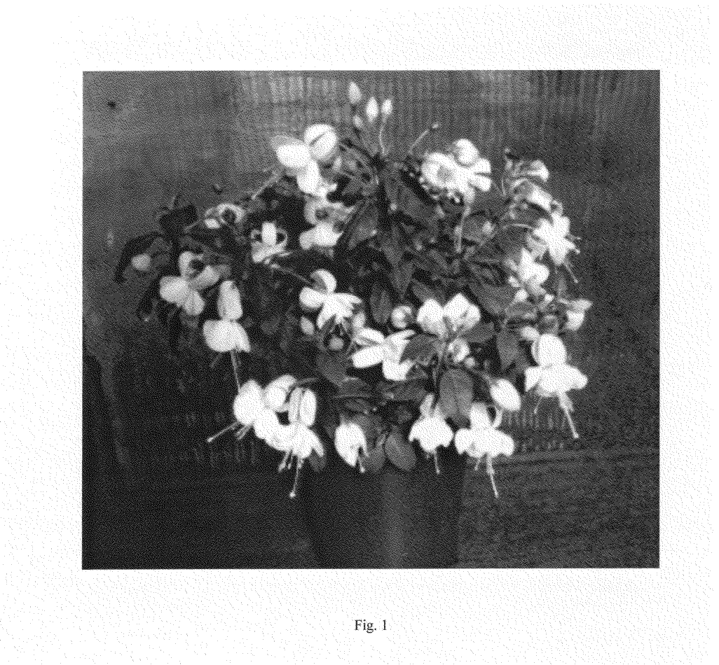

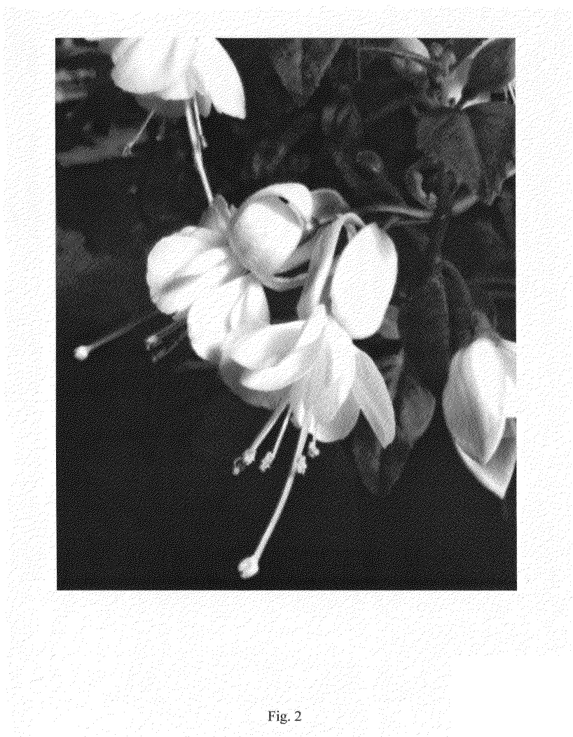

The accompanying photograph in FIG. 1 illustrates in full color a typical plant of ‘FWUWW22-0’ grown outdoors in Santa Paula, Calif. This plant is approximately 30 weeks old, shown in a 1 gallon pot. FIG. 2 illustrates a close up of the flowers. The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Colour Chart, 2001, except where general terms of ordinary dictionary significance are used. The following observations and measurements describe ‘FWUWW22-0’ plants in a commercial greenhouse in Santa Paula, Calif. Temperatures ranged from 10° C. to 29° C. night and day. No artificial light, photoperiodic treatments or chemical treatments were given to the plants. Natural light conditions were approximately 2500 to 4000 fc of light. Measurements and numerical values represent averages of typical plant types.
Botanical classification: Fuchsia hybrid ‘FWUWW22-0’
Propagation:
Propagation method: Vegetative Cuttings
Time to initiate roots: About 2 weeks at 15° C. to 22° C.
Root description: Fine, densely fibrous.
Plant:
Age of plant described: Approximately 30 weeks from a rooted cutting.
Pot size of plant described: 1 gallon
Height: Approximately 22 cm from soil line of pot to top of foliar and uppermost flowering plane.
Plant spread: Approximately 35 cm.
Growth rate: Rapid
Growth habit: Upright, compact
Branching characteristics: Free branching.
Length of primary lateral branches: Average 15 cm.
Diameter of lateral branches: Approximately 0.5 cm.
Quantity of primary lateral branches: Approximately 30
Characteristics of primary lateral branches:
   Color.—Near RHS Greyed-Red N181 C.
   Texture.—Glabrous.
   Strength.—Strong, flexible.
Internode length: Average 1.4 cm.
Foliage:
Leaf:
   Arrangement.—Opposite.
   Quantity.—Approximately 12 to 16 fully expanded leaves per primary lateral branch.
   Average length.—4.9 cm.
   Average width.—2.3 cm.
   Shape of blade.—Ovate.
   Apex.—Acute.
   Base.—Oblique.
   Margin.—Shallowly crenate.
   Texture of top surface.—Glabrous.
   Texture of bottom surface.—Glabrous.
   Aspect.—Slightly cupped upward along margins, and overall slight reflex downward over the entire leaf blade.
   Color.—Young foliage upper side: Near RHS Green 137A Young foliage under side: Near RHS Green 137B Mature foliage upper side: Near RHS Green 137A Mature foliage under side: Near RHS Green 137C.
   Venation.—Type: Pinnate. Venation color upper side: Near RHS Green 143C. Venation color under side: Near RHS Green 143D.
   Attachment.—Stalked.
   Petiole.—Length: Average 0.9 cm. Diameter: Average 0.15 cm. Texture: Pubescent. Color: Upper surface: Near RHS Yellow-Green 144A. Lower surface: Near RHS Green 138A.
Flower:
Natural flowering season: Year round under temperate conditions. Flowering is not dependent upon day length.
Inflorescence and flower type and habit: At least 2 flowers emerge singly from each axil. Flowers emerge at different times, resulting in flowers of different maturity at each axil. Flowers are symmetrical and composed of a corolla surrounded by a decorative fused calyx. Flowers face outwardly.
Flowers per plant: Approximately 45 fully open flower and 80 buds on a mature plant.
Flower longevity on plant: Approximately 1 week for individual flowers
Persistent or self-cleaning: Self-cleaning.
Flower size:
   Length.—Approximately 3.4 cm.
   Width.—Approximately 4.9 cm.
Bud:
   Shape.—Oblong, with an inflated sphere at terminal end.
   Length.—2.7 cm.
   Diameter.—1.6 cm.
   Color.—Near RHS Green-White 157A.
Corolla:
Petals:(Measured from separation at top of tube)
   Number.—4.
   Length.—Approximately 2.0 cm.
   Width.—Approximately 2.0 cm.
   Shape.—Broad deltoid to somewhat orbicular.
   Aspect.—Slightly upwardly cupped.
   Margin.—Entire.
   Texture.—Glabrous, all surfaces.
   Appearance.—Iridescent.
   Apex.—Obtuse.
Color:
   When opening.—Upper surface: Near RHS N155B, faint veins near Red-Purple 62D Lower surface: Near RHS N155B, faint veins near Red-Purple 62D.
   Fully opened.—Upper surface: Near RHS N155B, faint veins near Red-Purple 62D, but lighter. Lower surface: Near RHS N155B, faint veins near Red-Purple 62D, but lighter.
   Fading.—Upper surface: Near RHS N155B Lower surface: Near RHS N155B.
Calyx:
   Form.—Funnelform base, separating approximately 25% from base, to flare out into 4 large decorative deltoid shaped sepals.
   Fused section.—Length: Approximately 0.9 cm. Diameter: Approximately 0.7 cm.
   Unfused section.—Length: Approximately 2.8 cm. Diameter: Approximately 1.5 cm.
   Sepal texture.—Glabrous all surfaces.
   Appearance.—Irridescent.
   Apex.—Acute.
   Color.—Upper Surface: Near RHS White N155B, flushed Red-Purple 62D. Lower Surface: Near RHS White N155B, flushed Red-Purple 62D.
   Fragrance.—None.
Reproductive organs:
   Stamens.—Number (per flower): 8 Filament length: Average 2.5 cm Filament color: Near RHS Red-Purple 62D Anthers: Shape: Globular. Length: Approximately 0.1 cm. Color: Near RHS Orange-White 159C Pollen: Amount: Not present.
   Staminoid structures.—Number (per flower): 4 Length: Average 1.6 cm Color: Near RHS Red-Purple 62D.
   Pistils.—Quantity per flower: 1 Length: Approximately 5.2 cm Style: Length: Approximately 4.7 cm. Color: Near RHS Orange-White 159C Stigma: Shape: Linear Color: Near Red-Purple 62D Ovary: Length: Approximately 0.5 cm Diameter: Approximately 0.2 cm Color: Near RHS Yellow-Green 144C.
Other characteristics:
Seeds and fruits: Not observed to date.
Disease/pest resistance: Neither resistance nor susceptibility to normal diseases and pests of Fuchsia has been observed.
Temperature tolerance: Tolerates temperatures from approximately −1 C. to 38 C.
Drought tolerance: No tolerance for drought.

What is claimed is:

1. A new and distinct cultivar of Fuchsia plant named ‘FWUWW22-0’ as herein illustrated and described.
